(19) United States
(12) Patent Application Publication
(10) Pub. No.: US 2026/0306453 A1
(43) Pub. Date: Oct. 8, 2026

(54) FORMULATIONS CONTAINING TRYPTAMINE DERIVATIVES AND USES THEREOF

(71) Applicant: Psilera Inc., Tampa, FL (US)

(72) Inventors: Jacqueline L. Salm, Lutz, FL (US); Christopher G. Witowski, Tampa, FL (US)

(21) Appl. No.: 19/482,459

(22) PCT Filed: May 24, 2024
(86) PCT No.: PCT/US2024/030937

§ 371 Date: Nov. 7, 2025

Related U.S. Application Data
(60) Provisional application No. 63/504,034, filed on May 24, 2023.

(51) Int. Cl. A61K 31/4045; A61K 9/00; A61K 9/20; A61K 9/48

(52) U.S. Cl. CPC A61K 31/4045; A61K 9/0019; A61K 9/0043; A61K 9/2009; A61K 9/2013; A61K 9/2018; A61K 9/2027; A61K 9/2054; A61K 9/4858; A61K 9/4866

(57) ABSTRACT

Pharmaceutical compositions that comprise therapeutically effective amounts of a psychedelics may be used to treat, to manage or to prevent neurological, psychiatric and other diseases and disorders. Through delivery of these compounds, one may safely and effectively allow subjects to address the undesirable symptoms of these diseases and disorders and/or the diseases and disorders themselves.

### CROSS-REFERENCE TO RELATED APPLICATION

This patent application is a national stage application of PCT/US2024/030937, filed May 24, 2024, which claims the benefit of the filing date of U.S. provisional patent application Ser. No. 63/504,034, filed May 24, 2023; the entire disclosures of the afore-referenced patent applications are incorporated by reference.

### FIELD OF THE INVENTION

The present invention is directed to formulations of tryptamine derivatives and the uses of those formulations.

### BACKGROUND OF THE INVENTION

Psychedelic compounds are a group of compounds that affect one or more biologic activities such as perception, mood, and cognitive processes. There is great diversity in the structure and chemical activity of psychedelic compounds, but as a class they are known to elicit a broad spectrum neurological activity with rapid onset and durable duration of effects. Examples of well-known psychedelic compounds are psilocybin, N,N-dimethyltryptamine (DMT), 5-MeO-DMT, 4-AcO-DMT, lysergic acid diethylamide (LSD), and ibogaine, which mimic serotonin (5-HT), as well as target central nervous system (CNS) receptors.

Many psychedelic compounds have hallucinogenic effects that are attributed to serotonin receptor 2A (5-HT_{2A}) agonism. However, agonism alone, including partial agonism, does not dictate hallucinations, and the unique relationship between 5-HT_{2A} and metabotropic glutamate receptor 2 (mGlu2) deserve further investigation especially as it relates to addiction and compulsive behaviors. For example, signaling (such as G_{q} or beta-arrestin aggregation) has been shown to be important for downstream contribution to hallucinogenic effects. Additionally, as persons of ordinary skill in the art know, psychedelic compounds such as those identified in the previous paragraph target a variety of 5-HT receptors, of which there are fourteen subtypes, along with other CNS receptors (adrenergic, dopamine, histamine, muscarinic, opioid, sigma, and the like). Thus, although those compounds and others fall within the grouping known as psychedelics, their mechanisms for causing activity in the body are both diverse and complex.

Furthermore, although the aforementioned psychedelic compounds have been well-known for many years, recently there has been a move to develop new compounds in this class. For example, many novel compounds are described in WO 2021/226416 A1, Novel Compositions of Matter and Pharmaceutical Compositions, published Nov. 11, 2021. The search for new compounds, as well as desirable formulations for known compounds is driven by a goal of being able to better treat, prevent, and manage many diverse human conditions and disorders.

### SUMMARY OF THE INVENTION

The present invention is directed to pharmaceutical compositions that contain compounds that are tryptamine derivatives. The tryptamine derivatives are present in therapeutically effective amounts and have desirable biological activity and clinical safety.

In some embodiments, the present invention is a pharmaceutical composition, which may also be referred to as a formulation, that comprises, consists essentially of, or consists of a therapeutically effective amount of a compound that is defined by Structure (1) or a pharmaceutically acceptable salt, solvate, metabolite, or prodrug thereof:

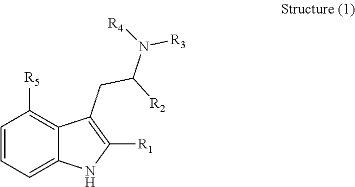

wherein R₁ is selected from the group consisting of Br, F, Cl, I, CF₃ and H;
R₂ is H or CH₃;
R₃ and R₄ are each independently selected from the group consisting of CH₃, C₂H₅, (H₃C)₂CH, H₂C═CH—CH₂ and H;
R₅ is selected from the group consisting of OCOCH₃, OH, OCH₃, O-phosphate, O-polyethylene glycol (PEG), O—(CH₂)₂(COOH)₂ (succinate), O—(CH₂)₂(COOH) (hemi-succinate), O-sulfate, fumarate, tartrate, maleate, sugar, amino acid, and CH₂SO₂NHCH₃ (sulfonamide); and
wherein when R₁ is H then R₅ is CH₂SO₂NHCH₃, CH₂NHSO₂CH₃, or CH₂NHCON(CH₂CH₃)₂.

In some embodiments, the compound from Structure (1) is selected from the group consisting of: 2-(2-bromo-1H-indol-3-yl)-N,N-dimethylethan-1-amine, 2-(2-chloro-1H-indol-3-yl)-N,N-dimethylethan-1-amine, 3-(2-(dimethylamino)ethyl)-1H-indol-4-yl acetate, 2-bromo-3-(2-(dimethylamino)ethyl)-1H-indol-4-yl acetate, N-(2-(2-bromo-1H-indol-3-yl)ethyl)-N-isopropylpropan-2-amine, N-(3-(2-(dimethylamino)ethyl)-1H-indol-4-yl)methanesulfonamide, and N-(3-(2-(dimethylamino)ethyl)-1H-indol-4-yl)acetamide.

In some embodiments, the formulation is a pharmaceutical composition that is designed for oral delivery into the human systemic circulation with quick onset and duration.

In some embodiments, the formulation is a pharmaceutical composition that is designed for extended release into the human systemic circulation via oral delivery, preferably providing a once daily dose e.g., for at least five days or at least 10 days. In some embodiments, the formulation is a pharmaceutical composition that is designed for extended release into the human systemic circulation via oral delivery, preferably providing a twice daily dose e.g., for at least five days or at least 10 days or at least 14 days. In some embodiments, the oral pharmaceutical compositions described herein are designed for modified time release of the active pharmaceutical ingredients, such as one or more compounds within Structure (1), into the human systemic circulation for extended duration.

In some embodiments, the pharmaceutical composition may, for example, comprise a solid, semisolid, liquid, or flexible delivery system and be delivered via sublingual, buccal, or oral administration. Additionally, the active pharmaceutical ingredient may, for example, be supplied within a tablet, capsule, softgel, strip, sublingual strip, wafer, solution, or suspension.

In some embodiments, the pharmaceutical compositions are designed for intermittent dosing on an every other day, a once-per-week, once-per-month, once yearly, or on an as needed basis.

In some embodiments, the pharmaceutical compositions are designed for injectable dosing by intraparietal (i.p.), intravenous (i.v.), intramuscular (i.m.), subcutaneous (s.c.), or implantable drug delivery devices.

In some embodiments, the pharmaceutical compositions are designed for topical or transdermal dosing via a patch, cream, ointment, gel, or lotion.

In some embodiments, the pharmaceutical compositions are designed for intranasal delivery via a liquid, gel, powder, nebulizer, spray, pneumatic delivery device, or pressurized delivery device.

In some embodiments, the compounds within the pharmaceutical compositions are 5-HT_{2A} partial agonists.

In some embodiments, the compounds within the pharmaceutical compositions are 5-HT_{2A} antagonists.

In some embodiments, the compounds within the pharmaceutical compositions are 5-HT_{2C} agonists.

In some embodiments, the compounds within the pharmaceutical compositions are 5-HT_{2B} agonists.

In some embodiments, the compounds within the pharmaceutical compositions are 5-HT_{5A} inhibitors.

In some embodiments, the compounds within the pharmaceutical compositions are 5-HT₆ agonists.

In some embodiments, the compounds within the pharmaceutical compositions are 5-HT_{7A} agonists.

In some embodiments, the compounds within the pharmaceutical compositions are 5-HT_{7A} inverse agonists.

In some embodiments, the compounds within the pharmaceutical compositions modulate sigma receptors.

In some embodiments, the compounds within the pharmaceutical compositions are non-hallucinogenic.

In some embodiments, the compounds within the pharmaceutical compositions are antidepressants.

In some embodiments, present invention provides a pharmaceutical composition that is effective for use in the treatment, management, or prevention of one or more of a neurological, mood, or impulse control disorder, including substance abuse disorders. In some embodiments, the disorders may be depression disorders, anxiety disorders, central nervous system inflammation associated with inflammatory disorders, Lyme disease, prion diseases, amoebic diseases, schizophrenia, schizo-affective disorders, psychotic depression disorders, bipolar depression disorders, refractory depressive disorders, Parkinson's Disease, impulse control disorders with addiction and habituating components, including, but not limited to: alcohol abuse disorders, nicotine dependence, opioid abuse disorder, stimulant abuse disorder, sedative hypnotic and tranquilizer dependence, gambling addiction, sex addiction, gaming technology disorder, attention deficit disorder, intermittent rage disorder, headache, post-traumatic stress disorder (PTSD), obsessive compulsive disorder, eating disorders, e.g. anorexia or bulimia, acute and/or chronic pain, amyotrophic lateral sclerosis, dementia of vascular or Lewy Body origin, frontotemporal dementias, Alzheimer's Disease, and organic disorders of cognition and memory.

In some embodiments, present invention provides a method of treatment, management, or prevention of one or more of a neurological, mood, or impulse control disorder, including but not limited to one or more of the aforementioned disorders.

In some embodiments, the present invention provides a medicament comprising a therapeutically effective amount of a compound of Structure (1). The medicament may, for example, be used to treat, to prevent, or to manage one of the aforementioned disorders.

In some embodiments, present invention provides use of a pharmaceutical composition comprising a therapeutically effective amount of a compound of Structure (1) for the prevention, treatment, or management of one or more of the aforementioned disorders.

In some embodiments, the pharmaceutical composition is capable of delivering the compound, which is an active ingredient or pharmaceutically acceptable salt or prodrug thereof. For example, the pharmaceutical composition may comprise an HBr salt of Structure (1) such as the HBr salt of 4-AcO-2-Br-DMT. In some embodiments, the present invention is directed to a pharmaceutical composition comprising a therapeutically effective amount of an HBr salt of Structure (1) such as the HBr salt of 4-AcO-2-Br-DMT. The therapeutically effective amount may, for example, be between about 0.01 and 35 mg/kg or 0.01 and 25 mg/kg or 0.01 and 20 mg/kg or between about 0.4 and 4 mg/Kg or between about 0.4 and 2 mg/Kg. This composition may, for example, be a medicament that is useful for the treatment of frontotemporal degeneration or dementia or other disorders or condition recited in this specification. In some embodiments, the composition comprises or is converted into a therapeutically effective amount of an active metabolite, e.g., 2-Br-4-OH-DMT, which is the active metabolite of 2-Br-4-AcO-DMT, which itself may be in the pharmaceutical composition that is administered or a salt of it such as the HBr salt may be in the pharmaceutical composition that is administered.

The compound may have biological activity that optionally may be defined by a satisfactory assessment from in vitro receptor screening and/or in vivo behavioral assessment in order to test for one or more of hallucinogenic effects, antidepressant effects, anxiolytic effects, pro-cognitive effects, learning, memory, post-traumatic stress disorder (PTSD) and alcohol use disorder. However, formulations within the scope of the present invention may additionally or alternatively show other neurological activity. Further, in some embodiments, the pharmaceutical compositions of the present invention may have therapeutic effects that include and/or are detectable by certain biomarkers. For example, they may have or affect neuroplastic properties associated with the biomarker brain-derived neurotrophic factor (BDNF). Other biomarkers of interest that may play a role in the mechanism of action of certain pharmaceutical compositions of the present invention include, but are not limited to, the inflammatory cytokines interleukin 6 (IL-6) and tumor necrosis factor alpha (TNFα), which are often elevated in patients with inflammatory diseases. Reducing these biomarkers in proinflammatory environments may reduce inflammation, especially neuroinflammation.

Through various embodiments of the present invention one may see one or more of improved in vitro receptor activity, functionality, dosage forms, and in vivo biological activity. Because of these benefits, various embodiments of the present invention may be desirable for therapeutic usage in humans to treat neurological and/or CNS disorders, as well as other disorders and conditions.

### BRIEF DESCRIPTION OF THE FIGURES

FIGS. 1A, 1B, 1C, and 1D are graphs that provide rat pharmacokinetic data.

FIGS. 2A and 2B are also graphs that provide rat pharmacodynamic data.

FIGS. 3A1 and 3A2 are graphs that measure the biomarker IL-6. FIGS. 3B1 and 3B2 are graphs that measure the biomarker BDNF. FIGS. 3C1 and 3C2 are graphs that measure the expression of TNF-alpha.

FIGS. 4A and 4B are graphs that measure the expression of mGlu2. FIG. 4C is a bar graph that shows a control relative to the expression of mGlu2.

FIG. 5 is a representation of the crystal structure of the HBr salt form of 2-Br-4-AcO-DMT.

FIG. 6 is a graph that measures the concentration of 2-Br-4-OH-DMT over time.

### DETAILED DESCRIPTION

Unless defined otherwise, all technical and scientific terms used herein have the same meaning as commonly understood by one of ordinary skill in the art.

As used in the specification and the accompanying claims the indefinite articles “a” and “an” and the definite article “the” include plural, as well as singular referents, unless the context clearly dictates otherwise.

The term “about” or “approximately” means an acceptable error for a particular value as determined by one of ordinary skill in the art, which depends in part on how the value is measured or determined. In some embodiments, the term “about” or “approximately” means within 1, 2, 3, or 4 standard deviations. In some embodiments, the term “about” or “approximately” means with 30%, 20%, 15%, 10%, 9%, 8%, 7%, 6%, 5%, 4%, 3%, 2%, 1%, 0.5%, 0.1%, or 0.05%, of a given value or range.

As used herein, and unless otherwise specified, the term “abuse disorder” is a disorder or disease that affects a person's brain and behavior and leads to an inability to control the use of a legal or illegal drug or medication. Prescription medicines, non-prescription medicines, and non-approved drugs may all be abused drugs. Drugs and medication may also include substances such as amphetamines, opioids, cocaine, barbiturates, alcohol, marijuana, and nicotine.

As used herein the term, and unless otherwise specified, an “Active Pharmaceutical Ingredient (API)” is any substance or mixture of substances intended to be used in the manufacture of a drug (medicinal) product and that, when used in the production of a drug, becomes an active ingredient of the drug product. Such substances are intended to affect the diagnosis, cure, mitigation, treatment, or prevention of disease or to affect the structure or function of the body.

The terms “co-administration,” “in combination with,” and “in combination” include the administration of two or more therapeutic agents either simultaneously, concurrently or sequentially within no specific time limits. In some embodiments, the agents are present in the cell or in the subject's body at the same time or exert their biological or therapeutic effect at the same time. In some embodiments, the therapeutic agents are in the same composition or unit dosage form. In other embodiments, the therapeutic agents are in separate compositions or unit dosage forms. Examples of co-administration agents include but are not limited to: antidepressants, anxiolytics, psychedelics, antipsychotics, anti-inflammatories, cognitive enhancing agents, beta amyloid reducing or tau modulating therapies, and/or enzyme inhibitors.

The terms “composition,” “formulation,” and “dosage form,” as used herein are intended to encompass compositions comprising the specified ingredient(s) (in the specified amounts, if indicated), as well as any product(s) that result, directly or indirectly, from combination of the specified ingredient(s) in the specified amount(s). By “pharmaceutical” or “pharmaceutically acceptable” it is meant that any diluent(s), excipient(s), absorption enhancer(s), or carrier(s) in the composition, formulation, or dosage form are compatible with the other ingredient(s) and not deleterious to the recipient thereof. Unless indicated otherwise, the terms “composition,” “formulation,” and “dosage form” are used herein interchangeably.

As used herein, the term “transdermal” relates to, being, or supplying a medication in a form for absorption through the skin into the bloodstream.

As used herein, the terms “intranasal” or “nasal” relates to, being or supplying a medication in a form for absorption through the nasal mucosa. Intranasal and nasal delivery may be affected through a wide range of dosage forms including but not limited to solutions, gels, suspensions, emulsions, liposomes and microparticles.

As used herein “immediate release” is defined as the formulation of an active pharmaceutical ingredient(s) drug taken orally, nasally, injected (i.p, i.v., or s.c.), or transdermally that results in the rapid absorption of the drug into the blood after administration. Immediate release may be measured in vitro using the FDA Industry guidance on dissolution and/or permeability testing, or in vivo using blood plasma levels.

As used herein, and unless otherwise specified, the terms “manage,” “managing” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a subject who had suffered from the particular disease in an attempt to prevent or minimize the recurrence of the disease.

As used herein, amelioration of the symptoms of a particular disorder by administration of a particular pharmaceutical composition refers to any lessening, whether permanent or temporary, lasting or transient, that can be attributed to or associated with administration of the composition.

As used herein, the term “metabolite” refers to an active form of a pharmaceutical that is administered to a subject in an inactive form. For example, a pharmaceutical may be administered as a prodrug in an inactive form, and after it is administered, a subject's body converts it into an active form that is capable of producing a desired effect.

As used herein, “modified release” or “extended release” is defined as a formulation of an active pharmaceutical ingredient(s) taken orally, nasally, injected, or transdermally that releases the active pharmaceutical ingredients over several hours or days, to maintain a relatively constant plasma concentration of the drug. Such modifications may have a number of objectives, such as maintaining therapeutic activity for an extended time, reducing toxic effects, protecting the active substance against degradation due to low pH, targeting the active substance to a predefined segment of the gastrointestinal tract for local treatment or targeting active substance release at specified time-points. Modified release is measured by the appropriate FDA industry guidelines on modified release formulations.

As used herein, and unless otherwise specified, the term “mood disorder” is a group of conditions where a disturbance in the person's mood is the underlying feature. Mood disorders may be groups of mania (elevated mood disorders) or hypomania (depression). The classification is in the Diagnostic and Statistical Manual of Mental Disorders (DSM) and the International Classification of Diseases (ICD).

As used herein, and unless otherwise specified, the term “neurological disorder” refers to diseases of the central and peripheral nervous system e.g., the brain, spinal cord, cranial nerves, peripheral nerves, nerve roots, autonomic nervous system, neuromuscular junction, and muscles. These disorders include epilepsy, Alzheimer's Disease, frontotemporal dementia and other dementias, and cerebrovascular diseases including, but not limited to, stroke, migraine, cluster headaches and other headache disorders, multiple sclerosis, Parkinson's Disease, viral and bacterial neuroinfections, post-acute sequelae of COVID, brain tumors, traumatic disorders of the nervous system due to head trauma, and traumatic disorders due to traumatic or terrifying experiences including, but not limited to, PTSD, and neurological disorders as a result of malnutrition and substance abuse. The substance abused may be any number of addictive substances, including, but not limited to, alcohol, other drugs and combinations thereof. Other embodiments are therapeutic and prophylactic compositions for central nervous system disorders associated with pathogenic bacterial species, including, but not limited to, Mycobacterial tuberculosis, Neisseria meningitides, Prevotella species, and spirochete species causing Lyme Disease. Additional embodiments are therapeutic and prophylactic compositions for central nervous system disorders associated with viruses, including, but not limited to, human herpes viruses, such as Epstein Barr Virus, Herpes Simplex Virus, as well as RNA viruses such as COVID-19, and Enteroviruses, in addition to lentiviruses such as Human Immunodeficiency Virus (HIV), and also those disorders associated with flaviviruses like West Nile Virus and Zika Virus.

As used herein “oral” relates to a medication in a form for absorption through the oral mucosal, sublingual, buccal, esophageal, gastric, or intestinal membranes. The term “capsule” refers to an oral composition in which the API and inactive ingredients are contained as a solid, liquid or semisolid within an outer shell comprised of gelatin, polymerized cellulose, or other suitable material. A capsule is intended to be swallowed wherein the composition will dissolve and release the API for systemic absorption through the esophageal, gastric, or intestinal lining.

The terms “strip” or “oral strip” include square, rectangular, triangular, rounded, circular or oblong shaped oral compositions that contain the API, inactive ingredients, and optionally a saliva stimulant, that form a pliable matrix. Upon entry into the mouth, commonly placed under the tongue, the composition will dissolve and release the API for systemic absorption through the buccal, sublingual, esophageal, gastric, or intestinal lining. Examples of formulation designed for orally delivering include, but are not limited to, those in the form of a tablet, capsule, softgels, strip, sublingual strip, wafer, solution, or suspension.

As used herein, and unless otherwise specified, the terms “prevent,” “preventing” and “prevention” refer to the prevention of the onset, recurrence or spread of a disease or disorder or of one or more symptoms thereof. In certain embodiments, the terms refer to the treatment with or administration of a compounder dosage form provided herein, with or without one or more other additional active agent(s), prior to the onset of symptoms, particularly to a subject at risk of diseases or disorders provided herein. The terms encompass the inhibition or reduction of a symptom of the particular disease. Subjects with familial history of a disease in particular are candidates for preventive regimes in certain embodiments. In addition, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder or prevent its recurrence. A prophylactically effective amount of a compound means an amount of therapeutic agent, alone or in combination with one or more other agent(s), that provides a prophylactic benefit in the prevention of the disease. The term “prophylactically effective amount” can encompass an amount that improves overall prophylaxis or enhances the prophylactic efficacy of another prophylactic agent.

Additional embodiments are therapeutic and prophylactic compositions for central nervous system disorders associated with fungal infections such as Cryptococcus and Aspergillus. Other additional embodiments are therapeutic and prophylactic compositions for central nervous system disorders associated with parasites including, but not limited to, those causing toxoplasmosis, malaria, Chagas Disease, and amoebic infections, which can affect the nervous system. Neurological symptoms may occur due to the infection itself, or due to an immune response to the pathogen or cytokines induced by the pathogens.

The term “subject” is defined herein to include animals such as mammals, including, but not limited to, primates (e.g., humans), cows, sheep, goats, horses, dogs, cats, rabbits, rats, mice and the like. In certain embodiments, the subject is a human.

The terms “tablet” and “wafer” includes spherical, round, oval, triangular, diamond, bullet, or oblong shaped oral compositions that contain the API, inactive ingredients, and optionally a saliva stimulant. Tablets and wafers may be formed by direct compression of a powdered formulation. Upon entry into the mouth, the compositions will dissolve and release the API for systemic absorption through the buccal, sublingual, esophageal, gastric, or intestinal lining.

As used herein, and unless otherwise specified, the terms “therapeutically effective amount,” “therapeutically effective dose,” and “effective amount” of a compound mean an amount sufficient to provide a therapeutic benefit in the treatment or management of a disease or disorder, or to delay or minimize one or more symptoms associated with the disease or disorder. A “therapeutically effective amount,” “therapeutically effective dose,” and “effective amount” of a compound mean an amount of a therapeutic agent, alone or in combination with one or more other agent(s), that provides a therapeutic benefit in the treatment or management of the disease or disorder. The terms “therapeutically effective amount,” “therapeutically effective dose,” and “effective amount” can encompass an amount that improves overall therapy, reduces or avoids symptoms or causes of disease or disorder, or enhances the therapeutic efficacy of another therapeutic agent.

As used herein, and unless otherwise specified, the terms “treat,” “treating” and “treatment” refer to the eradication or amelioration of a disease or disorder, or of one or more symptoms associated with the disease or disorder. In some embodiments, the terms refer to minimizing the spread or worsening of the disease or disorder resulting by the administration of one or more prophylactic or therapeutic agents to a subject with such disease or disorder. In some embodiments, the terms refer to the administration of a compound or formulation or dosage form provided herein, with or without one or more additional active agent(s), after the onset of symptoms of a particular disease.

In some embodiments, the pharmaceutical compositions of the present invention are used for treating neurological, mood and abuse disorders. These compositions comprising one or more compositions within Structure (1), and may be prepared for oral administration, transdermal administration, topical administration, intranasal administration or injectable administration. In certain embodiments, the pharmaceutical compositions comprise, consist essentially of or consist of pharmaceutically acceptable salts, solvates, or prodrugs of Structure (1) and combinations.

In some embodiments the active pharmaceutical ingredients are delivered in an oral pharmaceutical formulation composition comprising a capsule or tablet that delivers the API into the bloodstream through the esophageal, gastric, and/or intestinal membranes. By way of a non-limiting examples, the oral composition may be designed to be swallowed, and the active pharmaceutical ingredients are delivered into the bloodstream through the esophageal, gastric, and/or intestinal membranes. In some embodiments, the oral composition is in the form of a tablet, capsule, wafer, or strip that delivers the active pharmaceutical ingredient into the bloodstream through the sublingual, buccal, or other oral mucosal membrane.

In some embodiments, the oral composition comprises an oral patch or oral film that delivers the active pharmaceutical ingredient into the bloodstream through the sublingual, buccal, or other oral mucosal membrane.

In some embodiments, the oral composition comprises a powder, solution, or suspension that delivers the active pharmaceutical ingredient into the bloodstream through the sublingual, buccal, or other oral mucosal membrane.

In some embodiments, the pharmaceutical composition of the present invention is an immediate release formulation that releases the active pharmaceutical ingredient into the plasma upon oral administration. In some embodiments, the formulation comprises, consists essentially of, or consists of one or more active pharmaceutical ingredients of Structure (1) (or prodrugs or pharmaceutically acceptable salts or solvates thereof) in a therapeutically or prophylactically effective amount and optionally, one or more excipients.

In some embodiments, the pharmaceutical composition of the present invention is an extended or modified release formulation that effects a modified or extended release of the active pharmaceutical ingredient into the plasma upon oral administration. In some embodiments, the formulation comprises, consists essentially of, or consists of one or more active pharmaceutical ingredients of Structure (1) (or prodrugs or pharmaceutically acceptable salts or solvates thereof) in a therapeutically or prophylactically effective amount and optionally, one or more excipients.

In some embodiments, the pharmaceutical composition is designed for oral delivery and contains inactive ingredients that enhance drug delivery properties and stabilize the active ingredient. For example, one or more fillers may be included as inactive ingredients. The fillers can act as matrix to affect the dissolution time or act as binders to improve stability. Examples of fillers include, but are not limited to: starch, citric acid, tartaric acid, bicarbonates, phosphates, polyvinyl pyrrolidone, cellulose (natural and modified), croscarmellose, glycolates, acrylates, acetates, gelatin, gums, alginates, pectin, chitosan, chitin, salts, polysaccharides, mucilages, sugars, sucrose, lactose, and dextrose.

Additionally or alternatively, one or more lubricants may be included as inactive ingredients. The lubricants act to improve powder flowability or reduce friction on manufacturing parts. Examples of lubricants include, but are not limited to: magnesium stearate, talc, stearic acid, and silicon dioxide.

Additionally or alternatively, one or more flavorings may be included as inactive ingredients. The flavorings can mask the taste of bitter agents or improve the taste of the oral composition. Examples of flavorings include, but are not limited to: sugars, dextrose, sucrose, sucralose, stevia, essential oils, citric acid, and natural or artificial flavorings. Optionally, coloring agents can be included in the powders to improve visual properties or to differentiate product offerings; these coloring agents may, for example, be natural or artificial dyes, pigments, chelates, or metals.

Additionally or alternatively, one or more surfactants may be included in the composition. The surfactants may, for example, be emulsifiers and stabilizers that can encapsulate drugs for better stability, taste, permeability, and drug release properties. Surfactants include, but are not limited to, vegetable oils, triglycerides, esters, polysorbates (Tween), sorbitans (Span), phospholipids (e.g. lecithin), lauryl sulfates, betaines, propionates, fatty acids, fatty alcohols, saponins and alkanolamides, amine oxides, cyclodextrins, myristates and azones.

Additionally or alternatively, in some embodiments, the pharmaceutical composition is an oral formulation that contains co-solvents to improve drug solubility, dissolution, and permeability. Examples of co-solvents include, but are not limited to: alcohols such as ethanol, isopropanol, glycerin, propylene glycol, dipropylene glycol, polyethylene glycol, diethylene monoethyl ether, Cremophores, siloxanes, polyethylenes, and water.

Additionally or alternatively, in some embodiments, the pharmaceutical composition is an oral formulation that contains thickeners to reduce dissolution and provide a suitable matrix for delivery. Examples of thickeners include, but are not limited to: acrylates, carbomers, cellulose matrices, silicones, carrageenans, polysaccharides, and high melting point waxes and oils such as beeswax, coconut oil, palm oil, soybean oil, stearic acid, rapeseed oil, cocoa butter, shea butter, gums, rosins, resins, paraffins, and petroleum jelly.

Additionally or alternatively, in some embodiments, the pharmaceutical composition is an oral formulation that contains preservatives to improve formulation stability and retard microbial growth. Examples of preservatives include, but are not limited to: parabens, sorbates, benzoates, silicas, chlorides, phenols, chlorhexidine, citric acid, triclosan, Vitamin E (or tocopherols), chelators, metals, salts, and alcohols.

Additionally or alternatively, in some embodiments, the pharmaceutical composition is an oral formulation that contains enteric coatings to modify and extend release within the gastrointestinal tract. Examples of enteric coatings include, but are not limited to, high melting point waxes, fatty acids, sugars, fibers, and polymers and others.

Additionally or alternatively, in some embodiments, the pharmaceutical composition is an oral formulation that contains inactive ingredients that change the physical properties of the drug delivery system such as pH, solubility, dissolution, hydrophobicity, and stability. Many such compounds are known to persons of ordinary skill in the art.

Additionally or alternatively, in some embodiments, the pharmaceutical composition is an oral formulation that contains membrane penetration enhancers to increase systemic delivery. Examples of suitable penetration enhancers for use in the oral composition include, but are not limited to, C₂₋₄ alcohols, e.g., ethanol and isopropanol, polyethylene glycol monolaurate, polyethylene glycol-3-lauramide, dimethyl lauramide, sorbitans (Span), polysorbates (e.g., Tween, polysorbate 20), fatty acids, esters of fatty acids having from about 10 to about 20 carbon atoms, monoglycerides or mixtures of monoglycerides of fatty acids having a total monoesters content of at least 51% where the monoesters are those with from 10 to 20 carbon atoms, and mixtures of mono-, di- and tri-glycerides of fatty acids. Suitable fatty acids include, for example, lauric acid, myristic acid, stearic acid, oleic acid, linoleic acid and palmitic acid. Monoglyceride permeation enhancers include, for example, glycerol monooleate, glycerol monolaurate, and glycerol monolinoleate. Terpenes and terpenoids are derived from natural isoprene biosynthesis and can also be utilized to disrupt the skin membrane and increase API permeability. Examples of terpenes include, but are not limited to: menthol, menthone, camphor, nerolidol, limonene, myrcene, anethole, eugenol, 1,8-cineole, terpinolene, pinene, and humulene.

In some embodiments, the active pharmaceutical composition comprises at least one matrix in which the API resides. In some embodiments, the active pharmaceutical composition comprises at least two matrixes in which the API resides. In some embodiments, the active pharmaceutical composition comprises at least three matrixes in which the API resides. In some embodiments, the active pharmaceutical composition comprises at least four matrixes in which the API resides. In some embodiments, the active pharmaceutical composition comprises at least five matrixes in which the API resides.

In some embodiments, the pharmaceutical composition is designed to be delivered orally and is in the form of a patch, a powder, a suspension or a solution.

In some embodiments, the pharmaceutical compositions have single active ingredients. These may be delivered in the absence of other compositions that have active ingredients or co-administered with other compounds that are active ingredients that may or may not be within Structure (1). When there is co-administration, in some embodiments, a first agent, which may or may not be a pharmaceutical composition of the present invention may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, or 4 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, which may or may not be a pharmaceutical composition of the present invention.

In some embodiments, the pharmaceutical composition is designed to be administered orally and contains at least 1 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 45 mg, at least 50 mg, at least 60 mg, at least 70 mg, at least 80 mg, at least 90 mg, at least 100 mg, at least 120 mg, at least 140 mg, at least 160 mg, at least 180 mg, at least 200 mg, at least 250 mg, at least 300 mg, at least 350 mg, at least 400 mg, at least 450 mg, at least 500 mg, at least 750 mg, at least 1,000 mg, at least 1,250 mg, at least 1,500 mg, at least 1,750 mg, at least 2,000 mg, or not less than 2,000 mg of the active pharmaceutical ingredient of Structure (1).

In some embodiments, the pharmaceutical composition is designed to be administered orally and contains a therapeutic or prophylactic dose of at least 0.001 mg/kg, at least 0.01 mg/kg, at least 0.1 mg/kg, at least 1 mg/kg, at least 5 mg/kg, at least 10 mg/kg, at least 25 mg/kg, at least 50 mg/kg, at least 100 mg/kg, at least 150 mg/kg, at least 200 mg/kg, and/or not less than 200 mg/kg of the active pharmaceutical ingredient of Structure (1).

In some embodiments, the systemic drug release of the oral composition containing the active pharmaceutical ingredient occurs with therapeutically active onset of not more than 1 minute, not more than 3 minutes, not more than 5 minutes, not more than 7 minutes, not more than 9 minutes, not more than 11 minutes, not more than 13 minutes, not more than 15 minutes, not more than 17 minutes, not more than 19 minutes, not more than 21 minutes, not more than 23 minutes, not more than 25 minutes, not more than 27 minutes, not more than 30 minutes, not more than 45 minutes, not more than 60 minutes, not more than 90 minutes, not more than 120 minutes, not more than 150 minutes, or not more than 180 minutes.

In some embodiments, the duration of the oral composition containing the active pharmaceutical ingredient sustains for at least 5 minutes, at least 15 minutes, at least 30 minutes, at least 45 minutes, at least 60 minutes, at least 90 minutes, at least 120 minutes, at least 3 hours, at least 4 hours, at least 5 hours, at least 6 hours, at least 8 hours, at least 10 hours, at least 12 hours, at least 18 hours, at least 1 day, at least 2 days, at least 3 days, at least 4 days, at least 5 days, at least 6 days, at least 1 week, not less than 1 week, not less than 1 month, not less than 3 months, not less than 6 months, not less than 1 year, or at least once per life.

In some embodiments, the pharmaceutical composition is in the form of a solution to be delivered via i.p., i.v., i.m., s.c., or other form of implantable injection. The active pharmaceutical ingredient may be infused with inactive ingredients that solubilize and stabilize the active pharmaceutical ingredient for appropriate therapeutic delivery. The formulation also may contain a solvent that may, for example, include, but is not limited to: saline, water, methanol, ethanol, PEG, isopropyl alcohol, propylene glycol, dipropylene glycol, dimethylsulfoxide (DMSO), or functional derivatives thereof and combinations thereof.

In some embodiments, the injectable or implantable formulation uses oils or surfactants in order to solubilize, stabilize, or enhance the delivery of the active pharmaceutical ingredient. The oils may, for example, include fatty acids and oils that may be, but are not limited to: castor oil, coconut oil, medium chain triglycerides (MCT), jojoba oil, sunflower oil, argan oil, almond oil, olive oil, mineral oil, petroleum jelly, cocoa butter, shea butter, or other esters, triglycerides or functional derivatives thereof, polyethylene glycol monolaurate, polyethylene glycol-3-lauramide, dimethyl lauramide, monoglycerides or mixtures of monoglycerides of fatty acids having a total monoesters content of at least 51% where the monoesters are those with from 10 to 20 carbon atoms, and mixtures of mono-, di- and tri-glycerides of fatty acids. Suitable fatty acids include, for example, lauric acid, myristic acid, stearic acid, oleic acid, linoleic acid and palmitic acid. Monoglyceride permeation enhancers include, for example, glycerol monooleate, glycerol monolaurate, and glycerol monolinoleate. Examples of surfactants include, but are not limited to: polysorbates (e.g., Tween, polysorbate (20), sorbitans (Span), phospholipids (lecithin), lauryl sulfates, betaines, propionates, fatty alcohols and alkanolamides, fatty acid esters, amine oxides, myristates, and azones.

In some embodiments, the injectable or implantable formulation uses a stabilizer to protect the formulation or active pharmaceutical ingredient. Examples of these stabilizers include, but are not limited to, antibacterials, antifungals, antivirals, or similar drugs known by persons of ordinary skill in the art to retard the growth of microbial agents. Common preservatives include, but are not limited to: parabens, sorbates, benzoates, silicas, chlorides, phenols, chlorhexidine, citric acid, triclosan, Vitamin E (or tocopherols), chelators, metals, salts, and alcohols. Furthermore, a pH modifier can be used to enhance shelf life and stabilize the active pharmaceutical ingredient in the range of 0-2 pH, 2-4 pH, 4-6 pH, 6-8 pH, 8-10 pH, 10-12 pH, or 12-14 pH. Additionally, ionic charged agents known by those skilled in the art can be used to stabilize emulsified formulation of the active pharmaceutical ingredient.

In some embodiments the injectable or implantable formulation may be sterilized prior to or after packaging. Common examples of sterilization include, but are not limited to: gamma irradiation, ultraviolet light, ethylene oxide, hydrogen peroxide, peracetic acid, or filtration. The finished formulation may be sufficiently sterile of pathogens prior to injection into a subject, e.g., a patient, person, or animal in need.

In some embodiments, the injectable or implantable formulation contains at least 1 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 45 mg, at least 50 mg, at least 60 mg, at least 70 mg, at least 80 mg, at least 90 mg, at least 100 mg, at least 120 mg, at least 140 mg, at least 160 mg, at least 180 mg, at least 200 mg, at least 250 mg, at least 300 mg, at least 350 mg, at least 400 mg, at least 450 mg, at least 500 mg, at least 750 mg, at least 1,000 mg, at least 1,250 mg, at least 1,500 mg, at least 1,750 mg, at least 2,000 mg, or not less than 2,000 mg of an active pharmaceutical ingredient.

In some embodiments, the injectable or implantable formulation contains a therapeutic or prophylactic dose of at least 0.001 mg/kg, at least 0.01 mg/kg, at least 0.1 mg/kg, at least 1 mg/kg, at least 5 mg/kg, at least 10 mg/kg, at least 25 mg/kg, at least 50 mg/kg, at least 100 mg/kg, at least 150 mg/kg, at least 200 mg/kg, and/or not less than 200 mg/kg of the active pharmaceutical ingredient.

In some embodiments, the systemic drug release of the injectable or implantable formulation contains the active pharmaceutical ingredient and is designed such that therapeutically active onset is not more than 1 minute, not more than 3 minutes, not more than 5 minutes, not more than 7 minutes, not more than 9 minutes, not more than 11 minutes, not more than 13 minutes, not more than 15 minutes, not more than 17 minutes, not more than 19 minutes, not more than 21 minutes, not more than 23 minutes, not more than 25 minutes, not more than 27 minutes, not more than 30 minutes, not more than 45 minutes, not more than 60 minutes, not more than 90 minutes, not more than 120 minutes, not more than 150 minutes, and not more than 180 minutes.

In some embodiments, the duration of the therapeutic or prophylactic effect of the injectable or implantable formulation sustains for at least 5 minutes, at least 15 minutes, at least 30 minutes, at least 45 minutes, at least 60 minutes, at least 90 minutes, at least 120 minutes, at least 3 hours, at least 4 hours, at least 5 hours, at least 6 hours, at least 8 hours, at least 10 hours, at least 12 hours, at least 18 hours, at least 1 day, at least 2 days, at least 3 days, at least 4 days, at least 5 days, at least 6 days, at least 1 week, not less than 1 week, not less than 1 month, not less than 3 months, not less than 6 months, not less than 1 year, or at least once per life.

In some embodiments the topical or transdermal pharmaceutical dosage composition is in the form of sprayable liquids, gels, creams, lotions, ointments and transdermal patches, wherein the active pharmaceutical ingredient is infused with inactive ingredients that enhance the delivery properties of the composition and stabilize the active pharmaceutical ingredient. In one embodiment, penetration enhancers may be an inactive ingredient. The penetration enhancers may include fatty acids and oils that may be, but are not limited to: castor oil, coconut oil, medium chain triglycerides (MCT), jojoba oil, sunflower oil, argan oil, almond oil, olive oil, mineral oil, petroleum jelly, cocoa butter, shea butter, or other esters, triglycerides, or functional derivatives thereof.

In some embodiments, surfactants may be used in the topical or transdermal delivery system as emulsifiers, and stabilizers can be used to encapsulate drugs for better stability and permeability properties. Surfactants include, but are not limited to: polysorbates e.g. Tween, polysorbate (20), sorbitans (Span), phospholipids (lecithin), lauryl sulfates, betaines, propionates, fatty alcohols and alkanolamides, fatty acid esters, amine oxides, myristates, and azones.

In some embodiments, co-solvents may be used in the topical or transdermal formulation to improve drug solubility and permeability, while acting as a humectant for better skin feel. Common co-solvents include, but are not limited to: alcohols such as ethanol, isopropanol, glycerin, propylene glycol, dipropylene glycol, polyethylene glycol, diethylene monoethyl ether, Cremophores, siloxanes, polyethylenes, and water.

In some embodiments, thickeners may be used in the topical or transdermal formulation to reduce separation and provide a suitable matrix for modified delivery. Common thickeners include, but are not limited to: acrylates, carbomers, cellulose matrices, silicones, carrageenans, gums, resins, polysaccharides, and high melting point waxes and oils such as beeswax, coconut oil, palm oil, soybean oil, stearic acid, rapeseed, cocoa butter, shea butter, gums, rosins, resins, paraffins, and petroleum jelly.

In some embodiments, tackifiers may be used in the transdermal formulation to increase adhesion for extended wearability. Common tackifiers include, but are not limited to, gums, resins (natural or modified), carbomers, or other natural or synthetic polymers.

In some embodiments, preservatives may be used in the topical or transdermal formulation to improve formulation stability and retard microbial growth. Common preservatives include, but are not limited to: parabens, sorbates, benzoates, silicas, chlorides, phenols, chlorhexidine, citric acid, triclosan, Vitamin E (or tocopherols), chelators, metals, salts, and alcohols. Additionally or alternatively, the formulation may be emulsified with a hydrophilic ingredient such as water or Aloe barbadensis juice.

In some embodiments, the transdermal dosage form is a transdermal delivery device. Any device conventional in the art for transdermally delivering a therapeutic agent to a subject can be used for the transdermal delivery of the compositions of the invention and as the transdermal delivery device. For example, the transdermal delivery device can be a reservoir-type transdermal delivery device, a polymer-matrix type transdermal delivery device, or a drug-in-adhesive type transdermal delivery device or a multilaminate type transdermal delivery device. The transdermal delivery device may be designed so that when contacted with the subject's skin, the active pharmaceutical ingredient of the present invention is delivered in a therapeutically effective amount

In some embodiments, the transdermal delivery device is of the drug-in-adhesive type device comprising the active pharmaceutical ingredient dispersed directly in a pressure-sensitive adhesive matrix. The adhesive matrix may be supported on the topside with an impermeable backing film and on the side that faces the skin with an impermeable release liner. To administer the active pharmaceutical ingredient, the release liner is removed to expose the adhesive matrix, and the device is contacted with the skin. The adhesive matrix functions to adhere the device to the skin and, typically, to control the delivery rate of the active pharmaceutical ingredient. Similar to the polymer-matrix design, the drug-in-adhesive design allows the active pharmaceutical ingredient to diffuse out of the adhesive matrix, contact the subject's skin, and penetrate the skin. The delivery rate of the active pharmaceutical ingredient is usually determined by the rate of diffusion of the active pharmaceutical ingredient(s) out of the adhesive matrix. Multiple drug-in-adhesive layers can be laminated together between rate-controlling membranes for longer, extended delivery. The delivery rate is such that effective amount of the active pharmaceutical ingredient is delivered to the subject, e.g., patient, in need of the active pharmaceutical ingredient.

In some embodiments, a reservoir-type transdermal delivery device comprises a reservoir, usually a liquid, or semisolid located between an impermeable backing film and a rate-controlling membrane that is covered with a pressure-sensitive adhesive skin-contacting layer. The reservoir, which may be a solution or a dispersion, contains the composition of the invention. The transdermal delivery device may be supported by the impermeable backing film and the adhesive surface is protected by a release liner. To administer the active pharmaceutical ingredient of the present invention, the release liner is removed to expose the pressure-sensitive adhesive and the pressure-sensitive adhesive is contacted with the skin. The active pharmaceutical ingredient of the present invention is permeable through the rate-controlling membrane, and penetrates through it and the adhesive, contacts the skin, and then penetrates the skin. The delivery rate of the active pharmaceutical invention is usually determined by the rate that the active pharmaceutical ingredient penetrates the rate-controlling membrane.

In some embodiments, the transdermal delivery device is of the polymer-matrix design. In the polymer-matrix design, the API may be dispersed in a polymer matrix that controls its delivery rate. Preferably the polymer-matrix reservoir is supported on an impermeable backing layer. An adhesive layer is attached to the surface of the polymer matrix. To administer the active pharmaceutical ingredients, the release liner is removed to expose the polymer matrix and the ring of pressure-sensitive adhesive, and the device is contacted with the skin. The adhesive holds the device against the skin so that the polymer matrix directly contacts the skin. When the polymer matrix is contacted with the skin, the active pharmaceutical ingredient(s) diffuse out of the polymer matrix, contacts the patient's skin, and penetrates the skin. The delivery rate of the active pharmaceutical ingredients is usually determined by the rate of diffusion active pharmaceutical ingredient out of the polymer matrix.

Adhesives may comprise cross-linking monomeric units or sites that can be incorporated into the adhesive polymers. For example, cross-linking monomers can be incorporated into polyacrylate polymers. The cross-linking monomers may, for example, provide sites for cross-linking the polymer matrix after dispersing the API, into the polymer. Known adhesives comprise cross-linking monomers for polyacrylate polymers and include, for example, polymethacrylic esters of polyols such as butylene diacrylate, butylene dimethacrylate and trimethylol propane trimethacrylate, polyisobutylene type adhesives and silicone. Other monomers that provide cross-linking sites include allyl acrylate, allyl methacrylate, diallyl maleate, silyl ethers, and silanes. Monomers are then polymerized using methods known by those skilled in the art to comprise polyacrylate (acrylics), polysiloxane (silicones), or polyisobutylene (or other rubber) adhesive matrices containing cross-linkers, functional groups, or vinyl acetate to suspend, stabilize, and release the active pharmaceutical ingredient.

In some embodiments of the present invention, the transdermal delivery device may optionally include one or more penetration enhancers, which increase the rate at which the active pharmaceutical ingredients penetrate through the subject's skin. Preferably, the penetration enhancer penetrates the rate-controlling membrane or diffuses out of the polymer matrix or adhesive matrix so that it can contact the subject's skin and improve penetration of active pharmaceutical ingredient as defined herein through the subject's skin.

Suitable penetration enhancers for use in the transdermal delivery devices and compositions of the invention include, for example, C₂₋₄ alcohols, e.g., ethanol and isopropanol, polyethylene glycol monolaurate, polyethylene glycol-3-lauramide, dimethyl lauramide, polysorbates, sorbitans, fatty acids, esters of fatty acids having from about 10 to about 20 carbon atoms, monoglycerides or mixtures of monoglycerides of fatty acids having a total monoesters content of at least 51% where the monoesters are those with from 10 to 20 carbon atoms, and mixtures of mono-, di- and tri-glycerides of fatty acids. Suitable fatty acids include, for example, lauric acid, myristic acid, stearic acid, oleic acid, linoleic acid and palmitic acid. Monoglyceride permeation enhancers include, for example, glycerol monooleate, glycerol monolaurate, and glycerol monolinoleate.

Additionally or alternatively, one may use terpenes and terpenoids that are derived from natural isoprene biosynthesis. These can be utilized to disrupt the skin membrane and increase API permeability. Examples of terpenes include but are not limited to: menthol, menthone, camphor, nerolidol, limonene, myrcene, anethole, eugenol, 1,8-cineole, terpinolene, pinene, and humulene. In certain embodiments, the transdermal patches as described herein are used co-administered with penetration enhancers. In certain embodiments, the penetration enhancers may include oils that may be, but are not limited to: castor oil, coconut oil, medium chain triglycerides (MCT), jojoba oil, sunflower oil, argan oil, almond oil, olive oil, mineral oil, petroleum jelly, cocoa butter, and shea butter. Other penetration enhancers for use in transdermal patches include, for example, C₂₋₄ alcohols, e.g., ethanol and isopropanol, polyethylene glycol monolaurate, polyethylene glycol-3-lauramide, dimethyl lauramide, polysorbates (Tween), sorbitans (Span), fatty acids, esters of fatty acids having from about 10 to about 20 carbon atoms, monoglycerides or mixtures of monoglycerides of fatty acids having a total monoesters content of at least 51% where the monoesters are those with from 10 to 20 carbon atoms, and mixtures of mono-, di- and tri-glycerides of fatty acids. Suitable fatty acids include, for example, lauric acid, myristic acid, stearic acid, oleic acid, linoleic acid and palmitic acid. Monoglyceride permeation enhancers include, for example, glycerol monooleate, glycerol monolaurate, and glycerol monolinoleate.

In some embodiments, the active pharmaceutical ingredient may be delivered in a once-a-day topical or transdermal patch application. In some embodiments, the API may be delivered in less than 6 hours. In some embodiments, the API may be delivered over the course of 6-12 hours. In some embodiments, the API may be delivered over 12-24 hours. In some embodiments the API may be delivered over 24-48 hours. In some embodiments, the transdermal patch may be applied once every 2 days; once every 3 days; once every 4 days; once every 5 days; once every 6 days; or once every 7 days. The transdermal patch delivery rate options will facilitate a subject's dosing compliance while delivering a steady-state systemic safe and effective drug concentrations.

In some embodiments, the transdermal or topical composition contains at least 1 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 45 mg, at least 50 mg, at least 60 mg, at least 70 mg, at least 80 mg, at least 90 mg, at least 100 mg, at least 120 mg, at least 140 mg, at least 160 mg, at least 180 mg, at least 200 mg, at least 250 mg, at least 300 mg, at least 350 mg, at least 400 mg, at least 450 mg, at least 500 mg, at least 750 mg, at least 1,000 mg, at least 1,250 mg, at least 1,500 mg, at least 1,750 mg, at least 2,000 mg, or not less than 2,000 mg of the active pharmaceutical ingredient.

In some embodiments, the transdermal or topical composition contains a therapeutic or prophylactic dose of at least 0.001 mg/kg, at least 0.01 mg/kg, at least 0.1 mg/kg, at least 1 mg/kg, at least 5 mg/kg, at least 10 mg/kg, at least 25 mg/kg, at least 50 mg/kg, at least 100 mg/kg, at least 150 mg/kg, at least 200 mg/kg, and/or not less than 200 mg/kg of the active pharmaceutical ingredient.

In some embodiments, the pharmaceutical composition is in the form of a solution or gel to be delivered transmucosally via the nose, mouth, lungs, buccal, sublingual, rectal or delivery through another suitable membrane. The active pharmaceutical ingredient is infused with inactive ingredients that solubilize and stabilize the active pharmaceutical ingredient for appropriate therapeutic delivery. The formulation may contain a solvent. Examples of solvents include, but are not limited to: saline, water, methanol, ethanol, PEG, isopropyl alcohol, propylene glycol, dipropylene glycol, dimethylsulfoxide (DMSO), or functional derivatives thereof.

In some embodiments, the transmucosal formulation uses oils or surfactants in order to solubilize, stabilize, or enhance the delivery of the active pharmaceutical ingredient. The oils may, e.g., include fatty acids and oils that may be, but are not limited to: castor oil, coconut oil, medium chain triglycerides (MCT), jojoba oil, sunflower oil, argan oil, almond oil, olive oil, mineral oil, petroleum jelly, cocoa butter, shea butter, or other esters, triglycerides, or functional derivatives thereof, polyethylene glycol monolaurate, polyethylene glycol-3-lauramide, dimethyl lauramide, monoglycerides or mixtures of monoglycerides of fatty acids having a total monoesters content of at least 51% where the monoesters are those with from 10 to 20 carbon atoms, and mixtures of mono-, di- and tri-glycerides of fatty acids. Suitable fatty acids include, for example, lauric acid, myristic acid, stearic acid, oleic acid, linoleic acid and palmitic acid. Monoglyceride permeation enhancers include, for example, glycerol monooleate, glycerol monolaurate, and glycerol monolinoleate. Examples of surfactants include, but are not limited to: polysorbates (e.g. Tween, polysorbate (20), sorbitans (Span), phospholipids (lecithin), lauryl sulfates, betaines, propionates, fatty alcohols and alkanolamides, fatty acid esters, amine oxides, myristates, and azones.

In some embodiments, the transmucosal formulation uses a stabilizer to protect the formulation or active pharmaceutical ingredient. Examples of stabilizers include, but are not limited to antibacterials, antifungals, antivirals, or similar drugs known by those in the art to retard the growth of microbial agents. Additionally or alternatively, one may include preservatives. Common preservatives include, but are not limited to: parabens, sorbates, benzoates, silicas, chlorides, phenols, chlorhexidine, citric acid, triclosan, Vitamin E (or tocopherols), chelators, metals, salts, and alcohols. Furthermore, a pH modifier can be used to enhance shelf life and stabilize the active pharmaceutical ingredient in the range of 0-2 pH, 2-4 pH, 4-6 pH, 6-8 pH, 8-10 pH, 10-12 pH, or 12-14 pH. Additionally or alternatively, ionic charged agents known by those skilled in the art can be used to stabilize emulsified formulation of the active pharmaceutical ingredient.

In some embodiments, the transmucosal formulation uses a thickener to reduce formulation separation and provide a matrix for tailored drug delivery. Examples of thickeners include but are not limited to: acrylates, carbomers, cellulose matrices, silicones, carrageenans, polysaccharides, and high melting point waxes and oils such as beeswax, coconut oil, palm oil, soybean oil, stearic acid, rapeseed, cocoa butter, shea butter, gums, rosins, resins, paraffins, and petroleum jelly.

In some embodiments, the transmucosal formulation may be sterilized prior to or after packaging. Common examples of sterilization include but are not limited to: gamma irradiation, ultraviolet light, ethylene oxide, hydrogen peroxide, peracetic acid, or filtration. The finished formulation may be sufficiently sterile of pathogens prior to injection into a subject, e.g., patient, person, or animal in need.

In some embodiments, the transmucosal formulation can be paired with a device to facilitate delivery into the nose, mouth, lungs, buccal, sublingual, rectal or delivery through another suitable membrane. Examples of formulations and device include but are not limited to: sprays, liquids, gels, nebulizers, pneumatic delivery devices, or pressurized delivery device.

In some embodiments, the transmucosal formulation contains at least 1 mg, at least 2 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 45 mg, at least 50 mg, at least 60 mg, at least 70 mg, at least 80 mg, at least 90 mg, at least 100 mg, at least 120 mg, at least 140 mg, at least 160 mg, at least 180 mg, at least 200 mg, at least 250 mg, at least 300 mg, at least 350 mg, at least 400 mg, at least 450 mg, at least 500 mg, at least 750 mg, at least 1,000 mg, at least 1,250 mg, at least 1,500 mg, at least 1,750 mg, at least 2,000 mg, or not less than 2,000 mg of the active pharmaceutical ingredient.

In some embodiments, the transmucosal formulation contains a therapeutic or prophylactic dose of at least 0.001 mg/kg, at least 0.01 mg/kg, at least 0.1 mg/kg, at least 1 mg/kg, at least 5 mg/kg, at least 10 mg/kg, at least 25 mg/kg, at least 50 mg/kg, at least 100 mg/kg, at least 150 mg/kg, at least 200 mg/kg, and/or not less than 200 mg/kg of the active pharmaceutical ingredient.

In some embodiments, the systemic drug release of the transmucosal formulation occurs with therapeutically active onset of not more than 1 minute, not more than 3 minutes, not more than 5 minutes, not more than 7 minutes, not more than 9 minutes, not more than 11 minutes, not more than 13 minutes, not more than 15 minutes, not more than 17 minutes, not more than 19 minutes, not more than 21 minutes, not more than 23 minutes, not more than 25 minutes, not more than 27 minutes, not more than 30 minutes, not more than 45 minutes, not more than 60 minutes, not more than 90 minutes, not more than 120 minutes, not more than 150 minutes, or not more than 180 minutes.

In some embodiments, the duration of the therapeutic or prophylactic effect of the transmucosal formulation sustains for at least 5 minutes, at least 15 minutes, at least 30 minutes, at least 45 minutes, at least 60 minutes, at least 90 minutes, at least 120 minutes, at least 3 hours, at least 4 hours, at least 5 hours, at least 6 hours, at least 8 hours, at least 10 hours, at least 12 hours, at least 18 hours, at least 1 day, at least 2 days, at least 3 days, at least 4 days, at least 5 days, at least 6 days, at least 1 week, not less than 1 week, not less than 1 month, not less than 3 months, not less than 6 months, not less than 1 year, or at least once per life.

As described herein, certain formulations are useful in performing methods relating to differing dosage amounts and/or dosage periods; providing alternative pharmacokinetic profiles, pharmacodynamic profiles, and/or safety profiles; permitting long term maintenance therapies; and providing for the testing of new indications for psychedelics.

Provided herein are methods of preventing, managing, and treating neurological, mood or addictive disorders including but not limited to: depression, anxiety, central nervous system inflammation, Lyme disease, Prion disease, amoebic disease, schizophrenia, Parkinson's disease, addiction (alcohol, tobacco, opioid, nicotine, tobacco, stimulants, sedatives, gambling, sex, and/or technology) headache, PTSD, obsessive compulsive disorder, eating disorders, pain (acute or chronic), amyotrophic lateral sclerosis, frontotemporal dementia and other dementias, Alzheimer's disease (AD) or disorders of cognition and memory.

In some embodiments, the active pharmaceutical ingredients are co-administered with one or more therapeutic agents. The co-administered agent may, e.g., fall into the following classes of compounds: antipsychotics, antidepressants, anxiolytics, stimulants, reuptake inhibitors (SSRI or SSNRI), monoamine oxidase inhibitors (MAOI), cognitive-enhancing agents, tricyclic antidepressants, mood stabilizers, NMDA antagonists and 5-HT agonists or 5-HT antagonists.

In yet another embodiment, the compounds of Structure (1) either singularly or in mixtures, are co-administered with one or more therapeutic agents to reduce substance abuse. For the treatment of opioid addiction, other co-administered compounds can include: methadone, buprenorphine, naloxone, naltrexone, and the like. For the treatment of alcoholism, other co-administered compounds can include ethyl alcohol, disulfiram, naltrexone, acamprosate, benzodiazepines, and the like. For the treatment of nicotine addiction, other co-administered compounds can include low dose nicotine, Bupropion, Varenicline and the like.

By way of a non-limiting example, the compound may be a compound within Structure (1) or a pharmaceutically acceptable salt, a solvate, or a prodrug thereof, such as 2-(2-bromo-1H-indol-3-yl)-N,N-dimethylethan-1-amine; 2-(2-chloro-1H-indol-3-yl)-N,N-dimethylethan-1-amine; 3-(2-(dimethylamino)ethyl)-1H-indol-4-yl acetate; 2-bromo-3-(2-(dimethylamino)ethyl)-1H-indol-4-yl acetate; N-(2-(2-bromo-1H-indol-3-yl)ethyl)-N-isopropylpropan-2-amine; N-(3-(2-(dimethylamino)ethyl)-1H-indol-4-yl)methanesulfonamide; and N-(3-(2-(dimethylamino)ethyl)-1H-indol-4-yl)acetamide. Salts of these compounds include but are not limited to HBr salts. In some embodiments, the pharmaceutical composition is a tablet or a capsule that may be designed as an immediate release or extended release formulation.

In some embodiments, the pharmaceutical composition is designed so that the therapeutically effective amount is 5-25 mg/Kg in mice or 0.01-35 mg/kg in humans or 0.01-25 mg/kg in humans or 0.01-20 mg/Kg in humans or 0.1-10 mg/Kg in humans or 0.4-4 mg/Kg in humans or 0.4-2 mg/Kg in humans or 1-1.5 mg/Kg in humans. As persons of ordinary skill in the art are aware, human formulations may be designed based on dosages that have been effective in animals such as mice. See e.g., Nair, Anroop B. and Jacob, Sherry, A Simple Practice Guide for Dose Conversion Between Animals and Human, Journal of Basic and Clinical Pharmacy, pp. 27-31 (2016), which is incorporated by reference. By way of a non-limiting examples, the pharmaceutical composition may comprise 2-(2-bromo-1H-indol-3-yl)-N,N-dimethylethan-1-amine and be administered in a single dose or daily or twice daily for at least five days, at least seven days, at least ten days or at least fourteen days.

Table 9 provides an example of tablet formulations within the scope of the present invention.

TABLE 9

| Example Ingredients for Tablet | % w/w |
|---|---|
| Compound within Structure (1) | 5-10% |
| Ethanol | 2-6% |
| BHT | 0.1-2% |
| Povidone | 1-3% |
| Microcrystalline cellulose | 12-20% |
| Mannitol | 30-55% |
| Silicified microcrystalline cellulose | 15-25% |
| Silica | 0.1-2% |
| Magnesium stearate | 0.1-2% |

Table 10 provides an example of a specific tablet formulation

TABLE 10

| Example Ingredients for Tablet | % w/w |
|---|---|
| 2-(2-bromo-1H-indol-3-yl)-N,N-dimethylethan-1-amine | 8% |
| also referred to as 2-Br-4-AcO-DMT) | |
| Ethanol | 4% |
| BHT | 1% |
| Povidone | 2% |
| Microcrystalline cellulose | 16% |
| Mannitol | 47% |
| Silicified microcrystalline cellulose | 20% |
| Silica | 1% |
| Magnesium stearate | 1% |

Table 11 provides an example of a specific tablet formulation

TABLE 11

| Example Ingredients for Tablet | % w/w |
|---|---|
| 2-(2-bromo-1H-indol-3-yl)-N,N-dimethylethan-1-amine | 8% |
| Hydrobromide salt (also referred to as 2-Br-4-AcO-DMT * HBr) | |
| Ethanol | 4% |
| BHT | 1% |
| Povidone | 2% |
| Microcrystalline cellulose | 16% |
| Mannitol | 47% |
| Silicified microcrystalline cellulose | 20% |
| Silica | 1% |
| Stearic acid | 1% |

Table 12 provides an example of capsule formulations within the scope of the present invention.

TABLE 12

| Example Ingredients for | |
|---|---|
| Capsules | % w/w |
| Compound within Structure (1) | 15-20% |
| Microcrystalline cellulose | 75-85% |
| Stearic acid | 0.1-2% |

Table 13 provides an example of a specific capsule formulation

TABLE 13

| Example Ingredients for | |
|---|---|
| Capsules | % w/w |
| 2-Br-4-AcO-DMT | 17% |
| Microcrystalline cellulose | 82% |
| Stearic acid | 1% |

Table 14 provides an example of a specific capsule formulation

TABLE 14

| Example Ingredients for | |
|---|---|
| Capsules | % w/w |
| 2-Br-4-AcO-DMT * HBr | 17% |
| Microcrystalline cellulose | 82% |
| Stearic acid | 1% |

Table 15 provides an example of a specific capsule formulation

TABLE 15

| Example Ingredients for | |
|---|---|
| Capsules | % w/w |
| 2-Br-4-AcO-DMT * HBr | 17% |
| Microcrystalline cellulose | 83% |

### EXAMPLES

### Example 1—Assays

In vitro radioligand binding assays of various serotonergic and sigma receptors are shown in Table 1A and Table 1B.

TABLE 1A
Radioligand Binding in vitro Assays - Ki (nM)

| Structure | 5-HT1A | 5-HT1B | 5-HT1D | 5-HT1E | 5-HT2A | 5-HT2B |
|---|---|---|---|---|---|---|
| 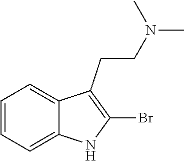 | >10000 | >10000 | >10000 | >10000 | 417.16 | 221.62 |
| 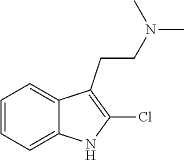 | >10000 | >10000 | >10000 | 3906.61 | 447.92 | 190.68 |
| 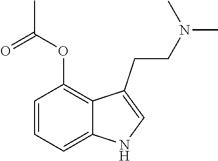 [SMILES: CC(=O)Oc1cccc2[nH]cc(CCN(C)C)c12] | >10000 | 2460.93 | 94.06 | 403 | 297.17 | 11.56 |
| 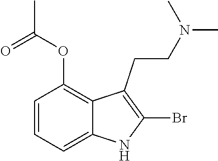 [SMILES: CC(=O)Oc1cccc2[nH]c(Br)c(CCN(C)C)c12] | >10000 | >10000 | >10000 | >10000 | 1127.2 | 67.69 |
| 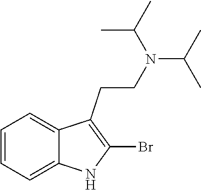 [SMILES: CC(C)N(CCc1c(Br)[nH]c2ccccc12)C(C)C] | >10000 | >10000 | 1072.26 | >10000 | >10000 | 45.21 |
| 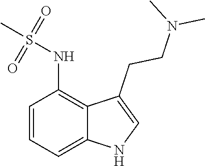 | 2900 | NT | NT | NT | >10000 | 1900 |
| 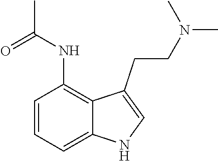 [SMILES: CC(=O)Nc1cccc2[nH]cc(CCN(C)C)c12] | 570 | NT | NT | NT | 3400 | 700 |
| N,N-dimethyltryptamine | >10000 | 557.06 | 42.24 | 104.06 | 185.91 | 44.47 |

TABLE 1B
Radioligand Binding in vitro Assays - Ki (nM)

| Structure | 5-HT2C | 5-HT5A | 5-HT6 | 5-HT7A | Sigma 1 | Sigma 2 |
|---|---|---|---|---|---|---|
| 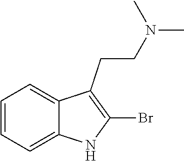 | 1294.49 | 1515.65 | 40.25 | 136.68 | 239.33 | 2458.67 |
| 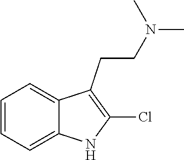 | 120.2 | 3001.93 | 62 | 177.99 | 637.68 | >10000 |
| 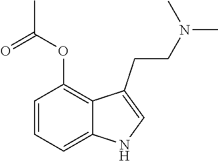 [SMILES: CC(=O)Oc1cccc2[nH]cc(CCN(C)C)c12] | 198.43 | 334.43 | 23.97 | 59.62 | >10000 | >10000 |
| 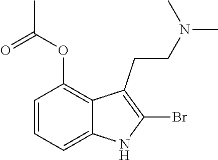 [SMILES: CC(=O)Oc1cccc2[nH]c(Br)c(CCN(C)C)c12] | 1216.47 | 893.72 | 70.71 | 396.46 | >10000 | >10000 |
| 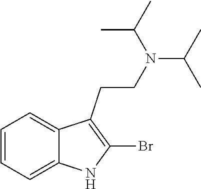 [SMILES: CC(C)N(CCc1c(Br)[nH]c2ccccc12)C(C)C] | >10000 | >10000 | 407.29 | 4932.87 | 256.98 | 482.84 |
| 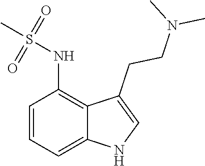 | >10000 | NT | >10000 | 930 | >10000 | >10000 |
| 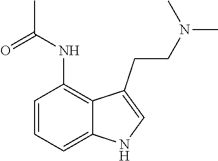 [SMILES: CC(=O)Nc1cccc2[nH]cc(CCN(C)C)c12] | 1000 | NT | >10000 | 290 | >10000 | >10000 |
| N,N-dimethyltryptamine | 138.39 | 154.63 | 133.51 | 15.23 | 229.99 | 776.6 |

The in vitro receptor pharmacology shows widespread serotonergic and sigma receptor activity with implications in various mental health and neurodegenerative disorders.

### Example 2—In Vitro Functional Activity

Table 2 below shows functional activity of certain compounds. The functional activity considers agonism vs. antagonism. The in vitro pharmacology for specific serotonergic receptors shows agonism at 5-HT_{2A}, 2A, 2C, and 6, but inverse agonism at 5-HT_{7A}. Each of these receptors affects mental health, learning, memory, and more specifically, 5-HT6 is responsible for precognitive effects and neuronal connections, growth, and migration.

TABLE 2

| | Agonism EC50 (nM) | | | | |
|---|---|---|---|---|---|
| Structure | 5-HT2A | 5-HT2B | 5-HT2C | 5-HT6 | 5-HT7A |
| 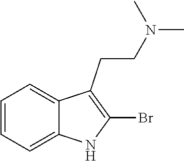 | >10000 | 799 | 2492 | 7 | 225 |
| 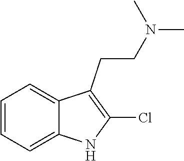 | 202 | 461 | 4479 | 9 | 630 |
| 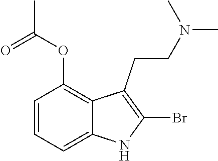 [SMILES: CC(=O)Oc1cccc2[nH]c(Br)c(CCN(C)C)c12] | 598 | 32 | 864 | 8 | 917 |
| 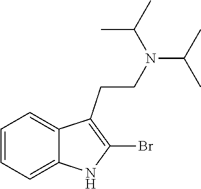 [SMILES: CC(C)N(CCc1c(Br)[nH]c2ccccc12)C(C)C] | 2131 | 18 | >10000 | 186 | 5866 |

### Example 3—Formulation Used in In Vivo Animal Experiments

Formulations for the compounds identified in example 2 were created. These formulations were used for i.p. injections in mice and p.o. oral formulations for PK/BBB in rats with ranges to show minimal side effects or excipient incompatibility. The final concentration of each compound was approximately 5 mg/mL. For each compound, a general formulation in the weight percent ranges shown in Table 3 was used:

TABLE 3

| Ethanol | PEG400 | Saline | Propylene Glycol | Tween 80 |
|---|---|---|---|---|
| <5% | <10% | 80-90% | 0% | 0% |
| 7-12% | 10-15% | 50-60% | 17-22% | 1-3% |
| 0% | 0% | 100% | 0% | 0% |

### Example 4—In Vivo Mouse Head-Twitch Response

The head-twitch response is a preclinical assay that tests for hallucinogenic effects in mice by counting the number of times a mouse will twitch its head relative to a negative control (vehicle) or positive control (hallucinogen, 4-AcO-DMT, a psilocin prodrug similar to psilocybin). The head twitch response data for each compound, provided in table 4 below, shows no statistically significant head twitches as compared to the formulation vehicle. This indicates that each tryptamine derivative is unlikely to cause hallucinations in humans caused by the 5-HT_{2A} receptor.

TABLE 4

| Structure | Head Twitch Response Results |
|---|---|
| 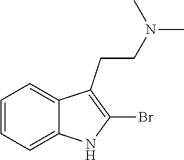 | No Head Twitch up to 50 mg/kg |
| 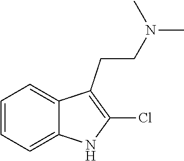 | No Head Twitch up to 100 mg/kg |
| 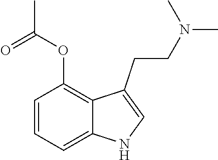 [SMILES: CC(=O)Oc1cccc2[nH]cc(CCN(C)C)c12] | Positive Control causes head twitch at 20 mg/kg |
| 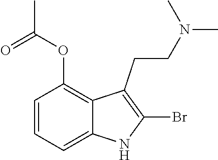 [SMILES: CC(=O)Oc1cccc2[nH]c(Br)c(CCN(C)C)c12] | No Head Twitch up to 50 mg/kg |
| 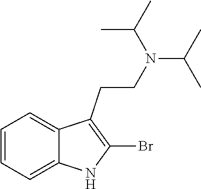 [SMILES: CC(C)N(CCc1c(Br)[nH]c2ccccc12)C(C)C] | No Head Twitch up to 25 mg/kg |
| 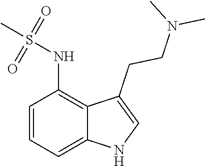 | No Head Twitch up to 25 mg/kg |
| 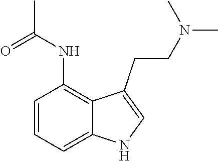 [SMILES: CC(=O)Nc1cccc2[nH]cc(CCN(C)C)c12] | No Head Twitch up to 50 mg/kg |

### Example 5—In Vivo Mouse Forced-Swim Test

The 2-day Porsolt Forced-Swim Test is a preclinical assay that tests for antidepressant effects in mice. Day 1 behavior is generally assumed to be related to resilience and longer swim times indicate an antidepressant effect similar to positive control (4-AcO-DMT). Day 2 behavior is more likely to assess learning and memory with shorter swim times indicative of learning to be rescued. Minimum effective doses for each active compound are provided in table 5, which shows that each of the compounds shown have longer swim times as compared to vehicle on Day 1 and shorter swim times compared to vehicle on Day 2 showing potential antidepressant effects as well as benefits to learning and memory.

TABLE 5

| Structure | Porsolt Forced Swim (Both Days) |
|---|---|
| 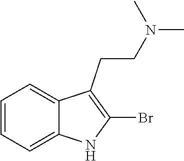 | 1-10 mg/kg |
| 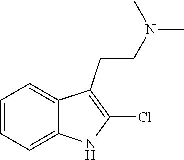 | 25 mg/kg |
| 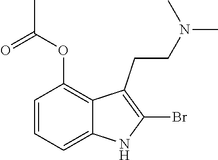 [SMILES: CC(=O)Oc1cccc2[nH]c(Br)c(CCN(C)C)c12] | 5 mg/kg |
| 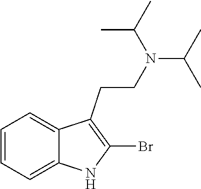 [SMILES: CC(C)N(CCc1c(Br)[nH]c2ccccc12)C(C)C] | 10 mg/kg |

### Example 6—In Vivo Mouse Elevated Plus Maze

The elevated plus maze is a preclinical assay that tests for anxiolytic effects in mice. Longer times or entries into the open arms indicate an anxiolytic effect similar to positive control (4-AcO-DMT). As table 6 shows, the results of the elevated plus maze indicate that each compound has potential anxiolytic effects beyond those shown by the placebo vehicle alone at the dose ranges shown. Each compound showed greater entries into arms as well as longer time in open arms as compared to vehicle.

TABLE 6

| Structure | Elevated Plus Maze |
|---|---|
| 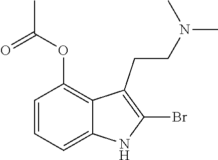 [SMILES: CC(=O)Oc1cccc2[nH]c(Br)c(CCN(C)C)c12] | 5-10 mg/kg |
| 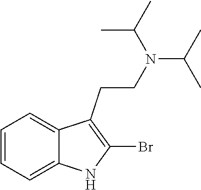 [SMILES: CC(C)N(CCc1c(Br)[nH]c2ccccc12)C(C)C] | 25 mg/kg |
| 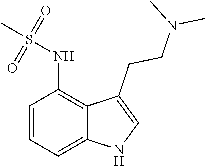 | 25 mg/kg |

### Example 7—In Vivo Mouse Fear Extinction and Conditioning

The fear extinction and conditioning model is a preclinical assay that performs a stimulus and tests response time and recognition of the stimulus. This translational model can correlate to anxiety, PTSD, memory, and precognitive effects to the positive control (4-AcO-DMT). As table 7 shows, each compound was shown to have a reduction in fear response in the fear extinction and conditioning mouse behaviors at the dose ranges shown indicating potential in anxiety, PTSD, learning and memory disorders.

Table 7

| Structure | Fear Extinction and Conditioning |
|---|---|
| 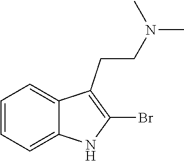 | 1-10 mg/kg |
| 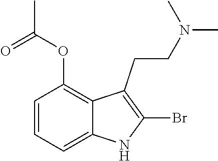 [SMILES: CC(=O)Oc1cccc2[nH]c(Br)c(CCN(C)C)c12] | 5-25 mg/kg |
| 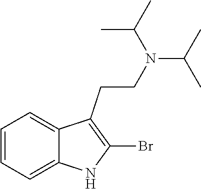 [SMILES: CC(C)N(CCc1c(Br)[nH]c2ccccc12)C(C)C] | 1-10 mg/kg |

### Example 8—In Vivo Mouse Reduction in Alcohol Consumption

In order to test for the ability to curb alcohol consumption, mice were given 8-20%/alcohol ab libitum for two weeks. The results of alcohol consumption were monitored over two weeks after daily dosing of test compound in relation to positive control (4-AcO-DMT). Further studies extended the experiment to 60 days for three different doses while only dosing the mice every 3 days. Alcohol consumption was similarly reduced at 25 and 35 mg/kg until nearly zero without signs of tolerance formation or negative effects over the 60 day period. As shown in table 8, the compound stops alcohol consumption in addicted mice at the dose ranges shown, indicating potential applications in human addiction such as alcohol use disorder.

TABLE 8

| Structure | Alcohol Consumption |
|---|---|
| 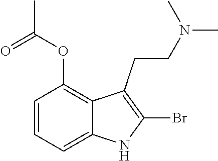 [SMILES: CC(=O)Oc1cccc2[nH]c(Br)c(CCN(C)C)c12] | 5-40 mg/kg |

### Example 9—Oral Vs Intravenous Pharmacokinetics

The formulation of Example 3 with 2-(2-bromo-1H-indol-3-yl)-N,N-dimethylethan-1-amine hydrobromide salt (also referred to as 2-Br-4-AcO-DMT*HBr) was used to establish a pharmacokinetic (PK) and metabolite profile.

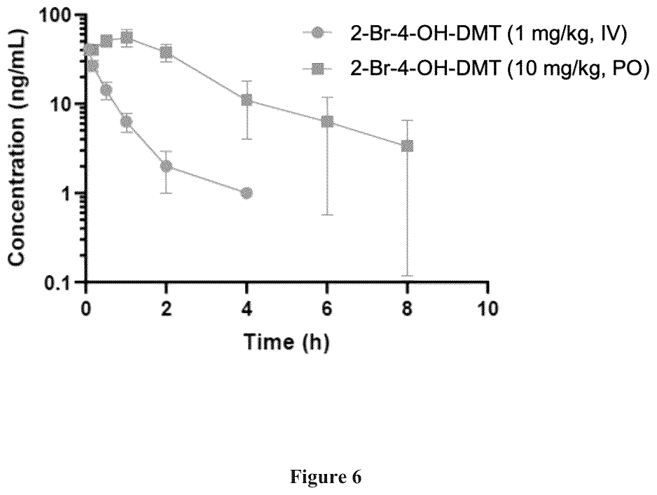

The rat PK (FIG. 6) was assessed with an oral dose of 10 mg/kg and intravenously at 1 mg/kg. 2-Br-4-AcO-DMT*HBr was rapidly metabolized via deacetylation into an active metabolite 2-bromo-3-(2-(dimethylamino)ethyl)-1H-indol-4-ol), also referred to as 2-Br-4-OH-DMT within minutes of oral or IV administration. The PK and brain concentrations were assessed in cohorts of 3 mice for each dosing group and between the plasma vs brain concentrations. For 10 mg/kg oral dosing, the PK parameters are outlined in Table 16 below. A 66% bioavailability was achieved via oral dosing of 2-Br-4-AcO-DMT*HBr upon conversion to 2-Br-4-OH-DMT.

TABLE 16
PK of 2-Br-4-OH-DMT after 10 mg/kg oral administration of 2-Br-4-AcO-DMT * HBr

| | | | | | | | AUC | | | | |
|---|---|---|---|---|---|---|---|---|---|---|---|
| 10 mg/kg, | t_{1/2} | Tₘₐₓ | Cₘₐₓ | AUCₗₐₛₜ | AUC_{Inf} | AUC/D | Extr | MRT | Vz_F | CL_F | F |
| PO | (h) | (h) | (ng/mL) | (h*ng/mL) | (h*ng/mL) | (h*kg*ng/mL/mg) | (%) | (h) | (L/kg) | (mL/min/kg) | % |
| Rat#1 | 2.33 | 1.00 | 68 | 220.49 | 244 | 24.40 | 9.63 | 3.40 | 137.50 | 683.13 | 86.97 |
| Rat#2 | 1.30 | 1.00 | 44 | 122.24 | 124 | 12.41 | 1.51 | 1.92 | 150.74 | 1342.85 | 48.22 |
| Rat#3 | 1.36 | 0.50 | 58 | 161.41 | 165 | 16.53 | 2.37 | 2.01 | 118.52 | 1008.11 | 63.67 |
| Mean | 1.66 | 0.83 | 57 | 168.05 | 178 | 17.78 | 4.50 | 2.44 | 135.59 | 1011.36 | 66.29 |
| SD | 0.58 | 0.29 | 12 | 49.46 | 61 | 6.09 | 4.46 | 0.83 | 16.20 | 329.87 | 19.51 |

1 mg/kg IV dosing, the PK parameters are outlined in Table 17.

TABLE 17
PK of 2-Br-4-OH-DMT after 1 mg/kg IV administration of 2-Br-4-AcO-DMT * HBr

| | | | | | | AUC | | | |
|---|---|---|---|---|---|---|---|---|---|
| 1 mg/kg, | t_{1/2} | C₀ | AUCₗₐₛₜ | AUC_{Inf} | AUC/D | Extr | MRT | Vss | CL |
| IV | (h) | (ng/mL) | (h*ng/mL) | (h*ng/mL) | (h*kg*ng/mL/mg) | (%) | (h) | (L/kg) | (mL/min/kg) |
| Rat#1 | 0.59 | 53 | 20.42 | 22 | 22 | 7.66 | 0.67 | 30.41 | 753.84 |
| Rat#2 | 0.71 | 62 | 24.38 | 25 | 25 | 4.06 | 0.79 | 31.01 | 655.81 |
| Rat#3 | 1.03 | 71 | 31.26 | 33 | 33 | 4.54 | 0.98 | 29.98 | 508.97 |
| Mean | 0.78 | 62 | 25.35 | 27 | 27 | 5.42 | 0.81 | 30.47 | 639.54 |
| SD | 0.23 | 9 | 5.49 | 5 | 5 | 1.96 | 0.16 | 0.52 | 123.24 |

The brain vs. plasma concentrations from oral and IV doses of 2-Br-4-ACO-DMT*HBr are listed in Table 18 via detection of active 2-Br-4-OH-DMT.

TABLE 18
Brain vs. plasma concentrations of 2-Br-4-OH-DMT after
oral and IV administration of 2-Br-4-AcO-DMT * HBr.

| | | | | Brain | Plasma | Brain / |
|---|---|---|---|---|---|---|
| | Dose | Time | Subject | Concentration | Concentration | Plasma |
| Route | (mg/kg) | (h) | No. | (ng/g) | (ng/mL) | Ratio |
| PO | 10 | 0.5 | Rat#1 | 1620 | 100 | 16.20 |
| | | | Rat#2 | 2205 | 102 | 21.62 |
| | | | Rat#3 | 3076 | 85 | 36.19 |
| | | | Mean | 2300 | 96 | 24.67 |
| | | | SD | 733 | 9 | 10.34 |
| | | 1 | Rat#4 | 2160 | 94 | 22.98 |
| | | | Rat#5 | 1906 | 66 | 28.88 |
| | | | Rat#6 | 3441 | 100 | 34.41 |
| | | | Mean | 2502 | 87 | 28.76 |
| | | | SD | 823 | 18 | 5.72 |
| | | 4 | Rat#7 | 453 | 16 | 28.31 |
| | | | Rat#8 | 798 | 28 | 28.50 |
| | | | Rat#9 | 519 | 20 | 25.95 |
| | | | Mean | 590 | 21 | 27.59 |
| | | | SD | 183 | 6 | 1.42 |
| IV | 1 | 0.5 | Rat#1 | 1600 | 33 | 48.48 |
| | | | Rat#2 | 746 | 41 | 18.20 |
| | | | Rat#3 | 980 | 42 | 23.33 |
| | | | Mean | 1109 | 39 | 30.00 |
| | | | SD | 441 | 5 | 16.21 |
| | | 1 | Rat#4 | 344 | 21 | 16.38 |
| | | | Rat#5 | 418 | 15 | 27.87 |
| | | | Rat#6 | 364 | 28 | 13.00 |
| | | | Mean | 375 | 21 | 19.08 |
| | | | SD | 38 | 7 | 7.79 |
| | | 4 | Rat#7 | 23 | 2 | 11.50 |
| | | | Rat#8 | 10 | BLOQ | NA |
| | | | Rat#9 | 10 | 1 | 10.00 |
| | | | Mean | 14 | 2 | 10.75 |
| | | | SD | 8 | 1 | 1.06 |

### Example 10——Nasal Formulation

The following formulation was created for intranasal delivery: 0.9% saline and 0.1% DMT fumarate.

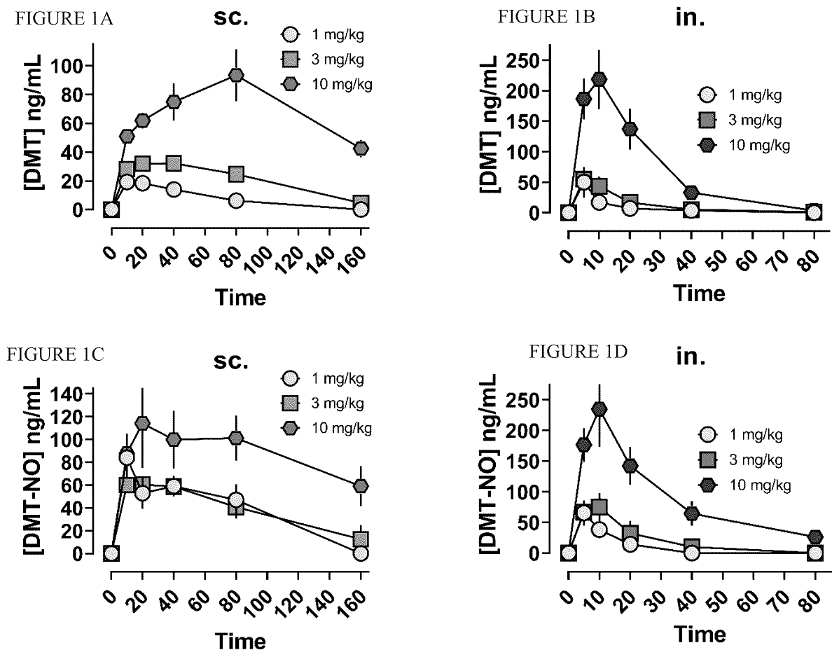

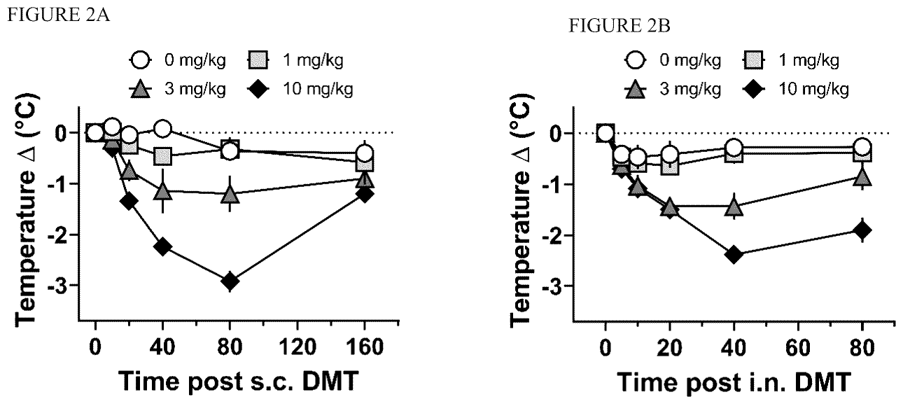

Rat PK and pharmacodynamic (PD) were collected. The rat PK (FIGS. 1A, 1B, 1C and 1D) and PD (FIGS. 2A and 2B) comparisons include intranasal formulation of DMT fumarate versus subcutaneous (s.c.) at three differences dosages. The concentrations detected of both DMT and its metabolite DMT-N-oxide (DMT-NO) were done via modified methods similar to Eckernas et al. (Journal of Pharmaceutical and Biomedical Analysis 2022.) There were six rats in each dose group, and they were restrained prior to dosing to allow for full nasal administration. The 3 mg/kg DMT i.n. dose in rats produces a similar Cₘₐₓ (60 ng/mL) to human bolus i.v. doses (FIGS. 1A-1D). Similar maximal effects (tₘₐₓ 10 mins) and duration (<60 min) were observed for i.n. as are seen in human i.v. doses. The i.n. administration of DMT also produced dose-dependent hypothermia as seen with similar studies on psychedelics (FIGS. 2A-2B). See Glatfelter, G. C. et al., ACS Pharmacology and Translational Science 2022, 5, 1181-1196, DOI: 10.1021/acsptsci.2c00177. The data indicates that these formulations provide desirable results when administered intranasally.

An intranasal (i.n.) delivery method for N,N-dimethyltryptamine (DMT) eliminates the need for the invasive delivery routes currently available in FDA clinical trials and avoids issues of related to the instability of DMT that may render some oral formulations such as capsules or tablets not viable delivery options. Additionally, intranasal delivery may be used to overcome the bioavailability challenges of that have been reported in connection with DMT formulations while still providing the rapid onset, duration, and necessary blood plasma levels for efficacious dosing.

### Example 11—CNS Biomarker Assessment of 2-Br-4-AcO-DMT (HBr Salt)

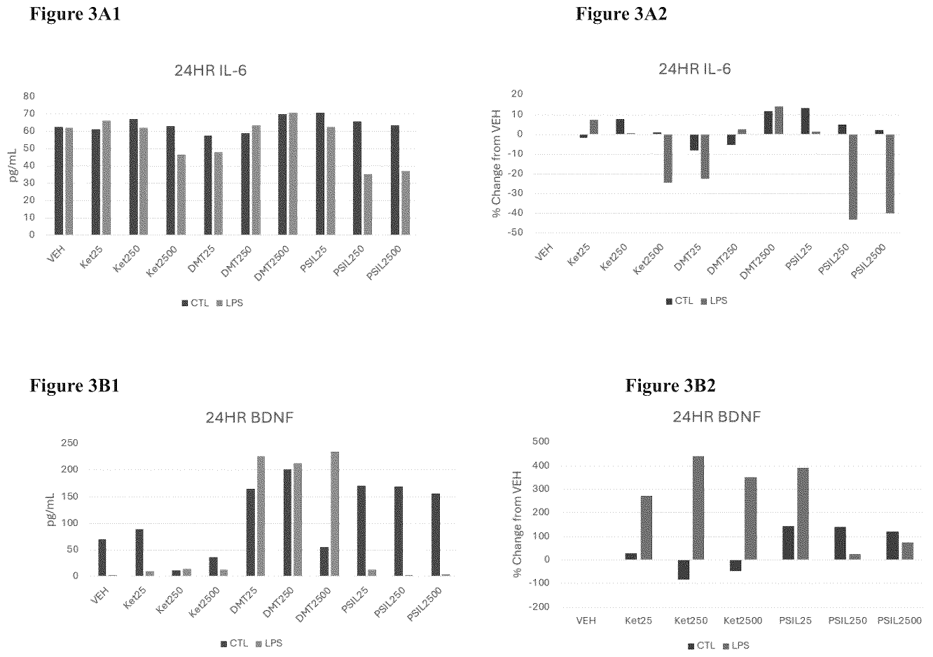

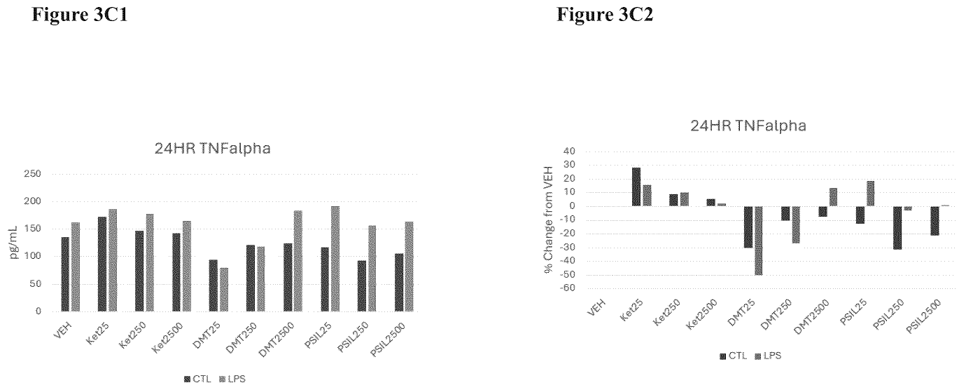

In order to assess the effects on neuroplasticity and inflammatory biomarkers, in vitro assays on immortalized mouse hippocampal neuronal cell line HT22 was cloned from HT4 cells and exposed to 2-Br-4-AcO-DMT HBr salt using ketamine and psilacetin as control compounds for 24 hours. For neuroplasticity, brain-derived neurotrophic factor (BDNF; FIGS. 3B1 and 3B2) and for inflammation, cytokine interleukin 6 (IL-6; FIGS. 3A1 and 3A2) and TNFα (FIGS. 3C1 and 3C2), were quantified with and without proinflammatory treatment of lipopolysaccharide (LPS). LPS-induced inflammation is a model for neuroinflammation similar to what is seen in patients with neurodegenerative disorders. Our data shows that 2-Br-4-AcO-DMT reduces IL-6 and TNFα in the stressed LPS cells (FIG. 3A1, 3A2, -3C1 and 3C2) and increases neuroplasticity marker BDNF in the control cells (FIG. 3B1 and 3B2). IL-6 is elevated in patients with frontotemporal dementias and other neurodegenerative disorders and especially in patients with disinhibition.

### Example 12—Brain Tissue Protein Expression in Mice after Treatment with 2-Br-4-AcO-DMT (HBr Salt)

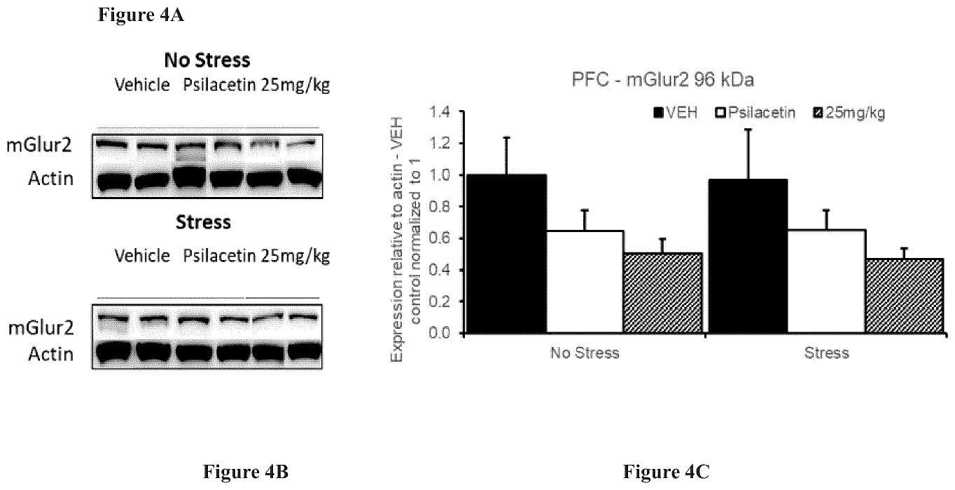

The expression of metabotropic glutamate receptor 2 (mGlu2) in the prefrontal cortex was quantified following in vivo mouse studies. Regulation of the metabotropic glutamate receptor 2 (mGlu2) levels in alcohol seeking rats can reduce relapse behavior. Following 18 days of daily treatment with vehicle, psilacetin, or 25 mg/kg 2-Br-4-AcO-DMT (denoted in FIGS. 4A-4C as 25 mg/kg), in either no-stress conditions or during 2 hour daily tube restraint, tissue was collected from multiple brain regions of male C57BL/6J mice. The mice treated with 2-Br-4-AcO-DMT showed reduced expression of mGluR2 in both the unstressed and stressed mice, (FIGS. 4A and 4B, 2 representative samples from each condition), exceeding the effect of psilacetin (n=5-6, data are calculated relative to actin, then normalized to vehicle (FIG. 4C). Data in FIG. 4C are represented as mean±SEM.

### Example 13—Crystal Structure of 2-Bru-4AcO-DMT Bromide Salt

In order to produce the HBr salt form of 2-Br-4-AcO-DMT, 4-AcO-DMT was placed in a flame dried round-bottom under argon. Anhydrous dichloromethane (DCM) was added to the flask. In a separate round-bottom flask, trimethylphenylammonium tribromide (PTT) was dissolved in anhydrous DCM under argon. The PTT solution was added dropwise to the reaction. The reaction stirred for 20 minutes and then was concentrated under reduced pressure. The crude product was loaded onto silica and purified using column chromatography 0-6% isopropyl alcohol in DCM. The pure product was subjected to isopropyl ether under reduced pressure to azeotrope and remove excess iPrOH. The product was finally subjected to freeze dryer conditions to remove trace amounts of water.

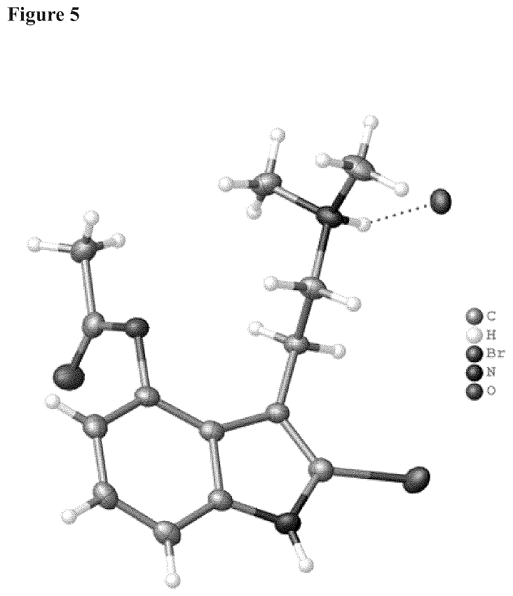

The product was isolated as the bromide salt as a grayish solid. FIG. 5 is a representation of the crystal structure, which is a monoclinic, P2₁/c and is centrosymmetric (non-chiral). Within the structure, molecules form chains through N—H . . . Br hydrogen bonds. The structure is further assembled through combination of weak interactions including C—H . . . O, C—H . . . pi, CH . . . Br and van der Waals. The structure contains four small voids (~10 A\hat{ }3 each) in unit cell with no electron density present.

1. A pharmaceutical composition comprising a therapeutically effective amount of a compound that is defined by Structure (1) or a pharmaceutically acceptable salt, a solvate, metabolite, or a prodrug thereof: 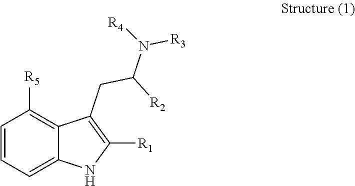
wherein R₁ is selected from the group consisting of Br, F, Cl, I, CF₃ and H;
R₂ is H or CH₃;
R₃ and R₄ are each independently selected from the group consisting of CH₃, C₂H₅, (H₃C)₂CH, H₂C═CH—CH₂ and H;
R₅ is selected from the group consisting of OCOCH₃, OH, OCH₃, O-phosphate, O-polyethylene glycol (PEG), O-succinate, O-hemi-succinate, O-sulfate, fumarate, tartrate, maleate, sugar, amino acid, and CH₂SO₂NHCH₃; and
wherein when R₁ is H then R₅ is CH₂SO₂NHCH₃, CH₂NHSO₂CH₃, or CH₂NHCON(CH₂CH₃)₂.

2. The pharmaceutical composition of claim 1, wherein R₁ is selected from the group consisting of Br, F, Cl, I, and CF₃.

3. The pharmaceutical composition of claim 2, wherein R₁ is Br.

4. The pharmaceutical composition of claim 2, wherein the composition is a tablet, the compound is the HBr salt of 4-AcO-2-Br-DMT, and the compound is approximately 5-10% by weight of the composition.

5. (canceled)

6. The pharmaceutical composition of claim 1, wherein R₅ is OCOCH₃.

7. (canceled)

8. (canceled)

9. (canceled)

10. The pharmaceutical composition of claim 1, wherein R₂ is H.

11. (canceled)

12. (canceled)

13. (canceled)

14. (canceled)

15. (canceled)

16. (canceled)

17. (canceled)

18. The pharmaceutical composition of claim 1, wherein R₃ is CH₃ and R₄ is CH₃.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The pharmaceutical composition of claim 1, wherein R₅ is OH.

51. The pharmaceutical composition of claim 2, wherein R₅ is OH, R₃ is CH₃ and R₄ is CH₃.

52. (canceled)

53. (canceled)

54. (canceled)

55. (canceled)

56. (canceled)

57. (canceled)

58. (canceled)

59. (canceled)

60. The pharmaceutical composition of claim 1, wherein the compound is 2-(2-bromo-1H-indol-3-yl)-N,N-dimethylethan-1-amine.

61. The pharmaceutical composition of claim 1, wherein the compound is 2-(2-chloro-1H-indol-3-yl)-N,N-dimethylethan-1-amine.

62. The pharmaceutical composition of claim 1, wherein the composition is a capsule, the compound is the HBr salt of 4-AcO-2-Br-DMT and the compound is approximately 15-20% by weight of the composition.

63. The pharmaceutical composition of claim 1, wherein the compound is 2-bromo-3-(2-(dimethylamino)ethyl)-1H-indol-4-yl acetate.

64. The pharmaceutical composition of claim 1, wherein the compound is N-(2-(2-bromo-1H-indol-3-yl)ethyl)-N-isopropylpropan-2-amine.

65. The pharmaceutical composition of claim 1, wherein the compound is N-(3-(2-(dimethylamino)ethyl)-1H-indol-4-yl)methanesulfonamide.

66. The pharmaceutical composition of claim 1, wherein the compound is N-(3-(2-(dimethylamino)ethyl)-1H-indol-4-yl)acetamide.

67. The pharmaceutical composition of claim 1, wherein the pharmaceutical composition is an oral delivery formulation.

68. The pharmaceutical composition of claim 1, wherein the pharmaceutical composition is an injectable delivery formulation.

69. The pharmaceutical composition of claim 1, wherein the pharmaceutical composition is an intranasal delivery formulation.

70-93. (canceled)

94. The pharmaceutical composition of claim 1 further comprising microcrystalline cellulose.
